(19) United States
(12) Patent Application Publication
(10) Pub. No.: US 2005/0251119 A1
(43) Pub. Date: Nov. 10, 2005

(54) Illuminated stylet

(75) Inventors: Leslie L. Eaton, Houston, TX (US); Donald A. Barr, League City, TX (US)

Correspondence Address: WONG, CABELLO, LUTSCH, RUTHERFORD & BRUCCULERI,;P.C., 20333 SH 249, SUITE 600, HOUSTON, TX, 77070, US

(73) Assignee: Muniz Engineering, Inc., Houston, TX (US)

(21) Appl. No.: 11/124,771

(22) Filed: May 9, 2005

Related U.S. Application Data
(60) Provisional application No. 60/569,661, filed on May 7, 2004.

(51) Int. Cl. A61B 18/18; F21V 8/00

(52) U.S. Cl. 606/15; 362/572

(57) ABSTRACT

An illuminated stylet includes a first fiber optic element for emitting a first light at its end and a second fiber optic element for emitting a second light along its length. The first and second light sources may be the same (e.g., white light) or different (e.g., red light and white light respectively).

### CROSS-REFERENCE TO RELATED APPLICATIONS

This application claims the benefit of U.S. Provisional Application Ser. No. 60/569,661 filed May 7, 2004, which is incorporated herein by reference in its entirety.

### FIELD OF THE PRESENT DISCLOSURE

The subject matter of the present disclosure relates to an apparatus and method of illuminating a stylet for intubating patients.

### BACKGROUND OF THE PRESENT DISCLOSURE

In some situation, illuminated stylets are useful for intubating patients. For example, illuminated stylets may be used when a patient has a difficult, obstructed or blocked airway, when conventional direct laryngoscopy has failed, or when circumstances do not permit use of laryngoscopy. During use, the illuminated stylet is inserted into an endotracheal tube until the light source at the distal end of the stylet reaches the tip of the tube. The tube and stylet can then be bent at a sharp angle to facilitate insertion into the oral cavity of a patient being intubated with their head in a neutral position. The end of the tube and stylet are then inserted into the patient's mouth, and the tip is advanced over the base of the tongue until a pre-tracheal glow is identified in the patient's neck. The pre-tracheal glow is a red, downward streaking glow seen in the neck of the patient from the lighted tip of the stylet. When the illuminated tip of the stylet is correctly positioned in the glottic opening, a bright red, teardrop shaped glow is illuminated in the anterior of the patient's neck. Identification of this pre-tracheal glow is used to determine proper insertion of the tube and stylet, and those performing the intubation look for characteristics of brightness, shape, and location to determine whether the tip is being properly inserted during the procedure.

Illuminated stylets currently in use have a single light source at the end of the stylet. Once the tip of the stylet passes the larynx, the oral cavity becomes dark, limiting the recognition of foreign objects or potential changes that can occur when the patient is choking, vomiting, or aspirating vomitus. In addition, producing the pretracheal glow requires translumination of the soft tissues in the neck, which may be hard for those performing the procedure to see when there is too much ambient light. Prior art illuminated stylets use white light for illumination. Because of the way the human eye perceives color, the red light portion of the white light is absorbed less so that more is transmitted through the skin and tissue to achieve the pre-tracheal glow due to the red hemoglobin in the blood and tissues. Conversely, the non-red components of the white light are absorbed by the tissues resulting in less light being transmitted completely through the bodies tissues to achieve the pretracheal glow. Furthermore, some prior art illuminated stylets, such as the Surch-lite stylet from Aaron Medical, have incandescent light bulbs on the tip of the stylet. Because of the size of these light bulbs, the stylet may not be suitable for use with endotracheal tubes having small internal diameters.

The subject matter of the present disclosure is directed to overcoming, or at least reducing the effects of, one or more of the problems set forth above.

### SUMMARY OF THE DISCLOSURE

An illuminated stylet includes an endotracheal tube, one or more light sources and one or more fiber optic elements. In one embodiment, the stylet has a fiber optic bundle positioned inside the tube and emitting red light substantially at a distal end of the tube. In another embodiment, the stylet has a bundle positioned inside the tube and has a scintillating fiber optic positioned outside the tube. The bundle emits light substantially at the distal end of the tube, while the scintillating fiber optic emits light substantially along a length of the fiber optic. The bundle and scintillating fiber optic can have the same light source or can have different light sources. In one embodiment, the bundle has a light source, such as a red laser diode, that generates red light at a wavelength of about 690-nm, while the scintillating fiber optic has a white light emitting diode (“LED”) as its light source.

### BRIEF DESCRIPTION OF THE DRAWINGS

The foregoing summary, preferred embodiments, and other aspects of the present disclosure will be best understood with reference to a detailed description of specific embodiments, which follows, when read in conjunction with the accompanying drawings, in which:

FIG. 1 is a side view of an embodiment of an illuminated stylet with a fiber optic bundle.

FIG. 2 is a side view of another embodiment of an illuminated stylet with an external scintillating fiber optic in addition to a fiber optic bundle.

FIGS. 3A-3C are end views of embodiments of fiber optic bundle, tubing, and second fiber optic element.

FIG. 4 is an exploded view of an embodiment of an LED lighting system for illuminating a single fiber bundle stylet.

FIG. 5 is an exploded view of an embodiment of an LED lighting system for an external scintillating fiber optic.

FIG. 6 is an exploded view of an embodiment of a laser diode module lighting system illuminated stylet with an external scintillating fiber optic in addition to a fiber optic bundle.

### DETAILED DESCRIPTION

An apparatus and method to illuminate a malleable stylet are disclosed. The apparatus includes a side-emitting fiber optic system attached to the stylet. Using the apparatus and method, manufacturers can incorporate the side-emitting fiber optic system during the construction of the stylet.

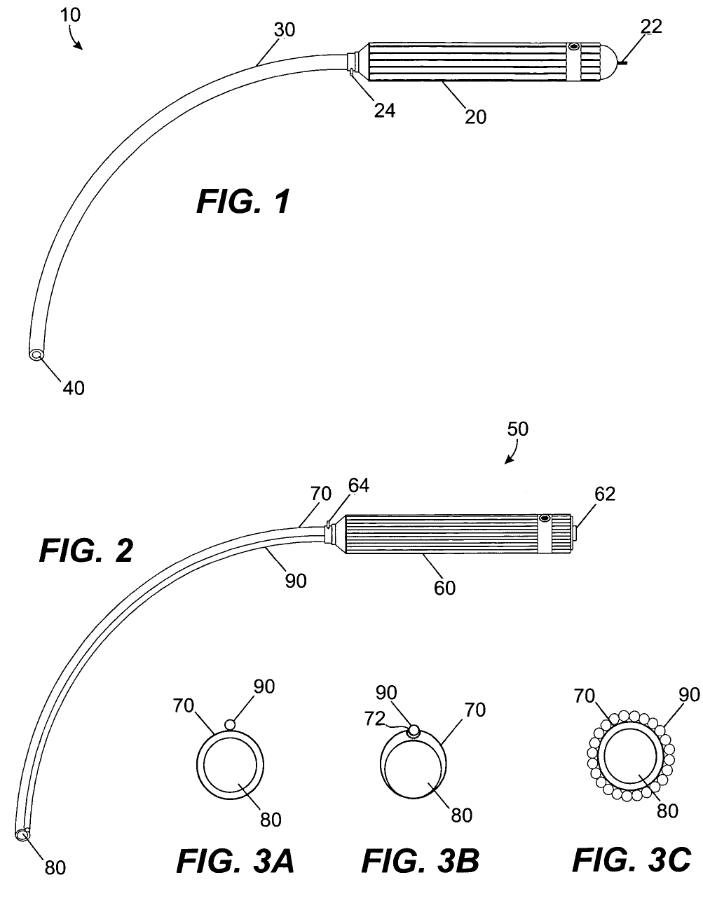

FIG. 1 is a side view of an embodiment of illuminated stylet 10 according to certain teachings of the present disclosure. Illuminated stylet 10 includes body or handle 20, endotracheal tube 30, and fiber optic element 40. A push button switch and end cap 22 insert into the end of handle 20. A set screw or other coupling mechanism 24 inserts into the opposing end of handle 20 and holds the malleable tubing of endotracheal tube 30 firmly into handle 20. In one embodiment, fiber optic element 40 is a fiber optic bundle coupled to a light source (not shown) housed in handle 20. Fiber optic bundle 40 is inserted into malleable tubing 30 of the stylet and is slightly shorter than the length of tube 30.

When the light source is activated by push button 22, the light source generates light, and fiber optic bundle 40 conveys the light to a distal end of bundle 40 where the light is emitted substantially at the distal end of tube 30. In one embodiment, the light source generates white light. In an alternative embodiment, the light source generates only red light and preferably red light having a wavelength around approximately 690-nm.

As evidence by the embodiment of FIG. 1, illuminated stylet 10 has a light source located in handle 20. The light source can be, for example, a high-intensity Light Emitting Diode (LED) or a laser diode. Light from the light source is transmitted through malleable tube 30 via multi-fiber bundle 40. This arrangement helps keep heat in the handle 20.

FIG. 2 is a side view of another embodiment of illuminated stylet 50 in accordance with the invention. Illuminated stylet 50 includes body or handle 60, endotracheal tube 70, first fiber optic element 80, and second fiber optic element 90. In one embodiment, first fiber optic element 80 is a fiber optic bundle, and second fiber optic element 90 is at least one scintillating fiber optic for emitting light from the side of endotracheal tube 70. Second side-emitting fiber optic 90 emits light throughout substantially the entire length of endotracheal tube 70. One illustrative scintillating fiber optic is the SparkleGlo product from PolyOptical Products, Inc., a division of Lumitex,Inc. Illumination from side-emitting fiber 90 can provide improved visualization of the oral cavity after the tip of tube 70 has passed the larynx. Both fiber optic systems 80 and 90 preferably provide the brightest light at their distal ends, facilitating visualization of pre-tracheal glow and proper placement of endotracheal tube 70 during use.

In one embodiment, elements 80 and 90 can share a common light source (not shown) housed in handle 60. In an alternative embodiment, each of elements 80 and 90 can have their own light source (not shown) housed in handle 60. These light sources can generate substantially the same characteristics of light. In yet another embodiment, multi-fiber bundle 80 is illuminated by a light source generating a different color of light than side emitting fiber optic 90, allowing for two different colors of light to be used to optimize visualization. For example, fiber optic bundle 80 can have a first light source that generates only red light, while scintillating fiber optic 90 can have a second light source that generates different light (e.g., white light).

As is known, light striking an object is absorbed, reflected, or transmitted through the object, and human perception of the wavelength of the light is indicated by color. To perceive a color correctly three primary elements must be present: red, green, and blue. The ratio of these primary elements gives a person the indication of the color of an object. The ratio of phosphors in a white LED is approximately one blue (470-nm), one red (690-nm) and two green (550-nm). This gives the perception of a white light. Due to the abundance of hemoglobin in blood, human tissue transmits the green and blue elements of the white light very poorly, yet red is transmitted readily. Thus, red or infrared is the color of choice for pulse oximetry. In a preferred embodiment, fiber optic bundle 80 has a light source generating red light with a wavelength of approximately 690-nm to facilitate pre-tracheal glow through the neck tissue of a patient, and scintillating fiber optic 90 has a light source generating white light. For example, central multi-fiber bundle 80 can have a red laser diode (690-nm) as its light source, and side-emitting fiber optic 90 can have a white LED light source. This color combination can improve visualization and can also efficiently use the battery power.

As shown, push button switch and end cap 62 inserts into the end of handle 60. A set screw or other coupling mechanism 64 inserts into the opposing end of handle 60 and holds the malleable tubing of endotracheal tube 70 of the stylet firmly into handle 60. Fiber optic bundle 80 is inserted into the malleable tubing of tube 70 and is slightly shorter than tube 70. Scintillating fiber optic 90 is positioned adjacent tube 70 along a length of tube 70. In the present embodiment, scintillating fiber optic 90 runs external to the malleable tubing of tube 70 (i.e., on the outside of tube 70) and is slightly shorter than the length of tube 70. In an alternative embodiment, scintillating fiber optic 90 can be inserted within tubing 70 along with fiber optic bundle 80.

As noted in the Background Section, prior art illuminated stylets use a single light source at the end of the stylet. Once the tip or distal end of the stylet passes the larynx, the oral cavity becomes dark, limiting the recognition of foreign objects or physical changes in a dynamic environment, e.g. the patient choking, vomiting, or aspirating vomitus. In contrast, the light emanating from side emitting fiber 90 in accordance with one embodiment of the invention provides better visualization of the oral cavity during the procedure.

Referring to FIG. 3A, in one embodiment of the invention external scintillating fiber optic 90 can be placed adjacent (e.g., piggybacked) to tube 70, while fiber optic bundle 80 can be positioned inside tube 70. As shown in FIG. 3B, an alternative embodiment has external scintillating fiber optic 90 run in groove 72 in tube 70 and can be covered with a medical-grade shrink tubing or clear coating (not shown) to provide firm attachment. As shown in yet another embodiment in FIG. 3C, second fiber optic element 90 can be a woven fiber optic mesh tube for the side-illuminated stylet. Malleable tube 70 and fiber optic bundle 80 can be inserted in the woven fiber optic mesh tube 90. This embodiment would allow for a full 360 degrees of light around the stylet for visualization in the oral cavity.

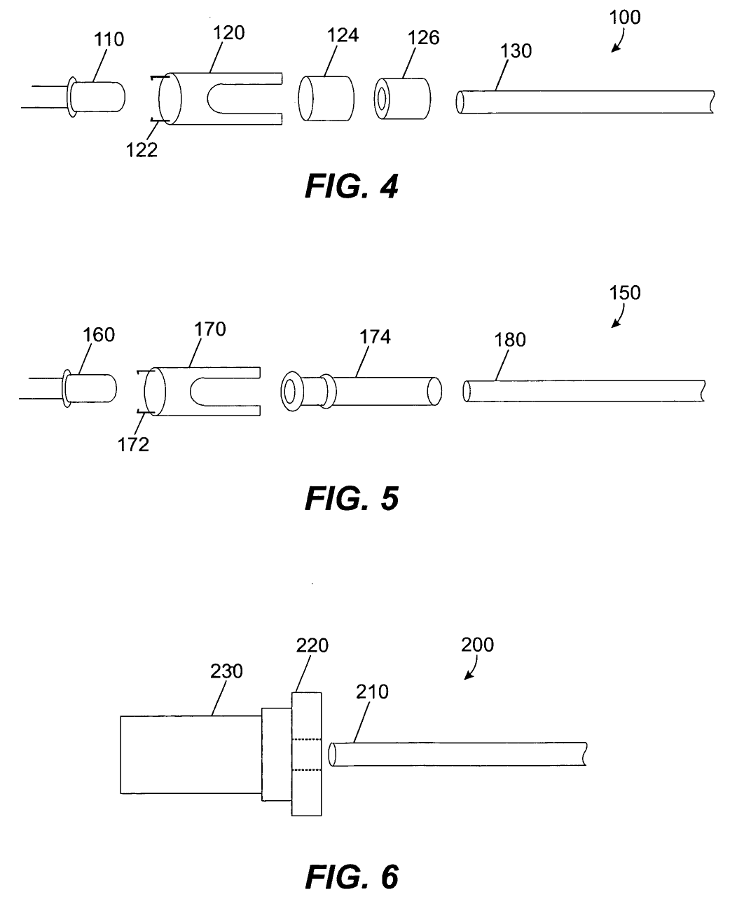

FIG. 4 shows an exploded view of an embodiment of LED lighting system 1 for illuminating a single fiber optic bundle stylet, such as in the embodiment of FIG. 1. As shown, LED 3 inserts into the end of plastic LED connector 6 that contains two small retaining legs 122. Brass tube 124 is friction fit onto styrene tube 126, and both ends of tube combination 124/126 are cut flush. Fiber optic bundle 9 is inserted through the center of styrene tube 126 until fiber bundle 9 is flush with the end of the styrene and brass tubes 124 and 126. The entire assembly of brass tube 124, styrene tube 126, and fiber optic bundle 9 is inserted into the U-shaped end of plastic LED connector 6 opposite LED 3.

FIG. 5 shows an exploded view of another embodiment of LED lighting system 13 using an external scintillating fiber optic, such as in the embodiment of FIG. 2. LED 15 inserts into one end of plastic LED connector 170 that contains two small retaining legs 172. Scintillating fiber optic 180 is inserted through the center of an unflared end of clear plastic ferrule 174 until fiber 180 is flush with the end of the flared end of ferrule 174. The entire assembly of ferrule 174 and fiber optic 180 is inserted into the U-shaped end of plastic LED connector 170 opposite LED 15.

FIG. 6 is an exploded view of an yet another embodiment of a laser diode module lighting system 16 for an illuminated stylet having an external scintillating fiber optic in addition to a fiber optic bundle, such as in the embodiment of FIG. 2. Scintillating fiber optic or multi fiber bundle 19 inserts into the end of laser diode module 21 attached to laser diode control board 23. Laser diode unit (21 and 23) can be custom built or purchased as an off-the-shelf component.

The disclosed illuminated stylets can be used with additional devices such as, for example, a laryngoscope. The lighting unit may be designed to be disposable with the stylet after a single use or reusable by removing and replacing the malleable tube and fiber optics. All components can be sterilized in situ with other parts of the stylet and endotracheal tube using gas autoclaves, ultraviolet light, or other conventional chemical sterilization techniques. The unit, as described herein, is battery operated and can be activated by a pull-tab for one-time use (i.e., for a disposable stylet) or activated by an on-off switch. For example, the lighting unit also can be designed to be reusable in two ways. In a first way, the malleable tube and fiber optics can be made removable and disposable so that only the handle and lighting unit housed in the handle are reused. In a second way, a disposable plastic sheath (similar to what is used for thermometers) can be used to cover the malleable tube.

The preceding description has been presented to enable any person skilled in the art to make and use the invention as claimed and is provided in the context of the particular examples discussed below, variations of which will be readily apparent to those skilled in the art. Accordingly, the claims appended hereto are not intended to be limited by the disclosed embodiments, but are to be accorded their widest scope consistent with the principles and features disclosed herein.

1. A medical stylet, comprising:
one or more light sources;
a first fiber optic element having a first end and a second end, wherein the first fiber optic element is coupled to at least one of the one or more light sources at the first end and adapted to emit light substantially from the second end; and
a second fiber optic element having a proximal end and a length, wherein the second fiber optic element is coupled to at least one of the one or more light sources at the proximal end and adapted to emit light substantially along the length.

2. The stylet of claim 1, wherein the first fiber optic element and the second fiber optic element are coupled to the same light source.

3. The stylet of claim 1, wherein the first fiber optic element is coupled to a first light source and the second fiber optic element is coupled to a second light source.

4. The stylet of claim 3, wherein the first light source is adapted to emit light at a first wavelength and the second light source is adapted to emit light at a second wavelength.

5. The stylet of claim 3, wherein the first light source comprises a red light source.

6. The stylet of claim 3, wherein the second light source comprises a white light source.

7. The stylet of claim 1, wherein the second fiber optic element comprises at least one scintillating fiber optic.

8. The stylet of claim 1, wherein the second fiber optic element comprises a fiber optic mesh.

9. The stylet of claim 1, further comprising a tube, wherein the first fiber optic element is positioned with the tube and the second fiber optic element is positioned outside and adjacent the tube.

10. The stylet of claim 9, wherein the second fiber optic element is positioned in a groove defined in an outside surface of the tube.

11. The stylet of claim 9, wherein the second fiber optic element comprises a scintillating fiber optic.

12. The stylet of claim 9, wherein the second fiber optic element comprises a fiber optic mesh.

13. The stylet of claim 1, wherein the at least one of the one or more light sources comprise a light emitting diode.

14. The stylet of claim 1, wherein at least one of the of the one or more light sources comprise a laser diode.

15. The stylet of claim 1, further comprising a power source coupled to the one or more light sources.

16. A stylet for use with an endotracheal tube, the tube having a proximal end and a distal end, the stylet comprising:
a first light source positioned substantially adjacent to the proximal end of the tube;
a first fiber optic element positioned inside the tube and coupled to the first light source at a first end and adapted to emit light from the first light source at a second end;
a second light source positioned substantially adjacent to the proximal end of the tube; and
a second fiber optic element positioned outside the tube and coupled to the second light source at a first end and adapted to emit light along a length of the second fiber optic element.

17. The stylet of claim 16, wherein the first light source comprises a red light source and the second light source comprises a white light source.

18. The stylet of clam 16, wherein the first light source and the second light source comprise the same light source.

19. The stylet of claim 16, wherein the second fiber optic element comprises a scintillating fiber optic element.

20. The stylet of claim 16, further comprising a power source coupled to the first and second light sources.

21. The stylet of claim 20, wherein the power source comprises one or more batteries.

22. The stylet of claim 16, wherein at least one of the light sources comprise a light emitting diode.

23. The stylet of claim 16, wherein at least one of the light sources comprise a laser diode.

24. A stylet for use with an endotracheal tube, the tube having a proximal end and a distal end, the stylet comprising:
first light means for emitting a first light;
first emitting means for emitting the first light substantially at the distal end of the tube;
second light means for emitting a second light; and
second emitting means for emitting the second light from the proximal end of the tube to the distal end of the tube.

25. The stylet of claim 24, wherein the first light means comprises a means to emit substantially red light.

26. The stylet of claim 25, wherein the second light means comprises a means to emit substantially white light.

27. The stylet of claim 24, further comprising a power source coupled to the first and second light means.

28. The stylet of claim 24, wherein the first and the second light means comprise the same light means.
